(19) United States
(12) Patent Application Publication
(10) Pub. No.: US 2021/0402446 A1
(43) Pub. Date: Dec. 30, 2021

(54) AUTOMATED MOBILE DEVICE FOR CLEANING AND DISINFECTING

(71) Applicant: Piedmont National Corporation, Atlanta, GA (US)

(72) Inventors: John David Thompson, Woodstock, GA (US); David Drew Thompson, Ball Ground, GA (US); James Douglas Vaughan, Greenville, SC (US); Benjamin Powers Boney, Knoxville, TN (US); John Edward Garlock, Atlanta, GA (US)

(73) Assignee: Piedmont National Corporation, Atlanta, GA (US)

(21) Appl. No.: 16/948,366

(22) Filed: Sep. 15, 2020

Related U.S. Application Data
(60) Provisional application No. 63/044,875, filed on Jun. 26, 2020.

(51) Int. Cl. B08B 7/00; G05D 1/00; G05D 1/02; A61L 2/24; A61L 2/10

(52) U.S. Cl. CPC B08B 7/0057; G05D 1/0094; G05D 1/0251; G05D 1/0219; A61L 2/24; A61L 2202/14; G05D 2201/0203; A61L 2202/17; A61L 2202/16; A61L 2202/11; A61L 2/10

(57) ABSTRACT

A cleaning system includes a mobile base that enables movement of the cleaning system. The cleaning system also includes a camera that captures an image of a cleaning location and an ultraviolet light source that transmits a disinfecting light output toward the cleaning location. Additionally, the cleaning system includes a processor and a memory. The memory includes instructions stored thereon that are executable by the processor to perform operations. The operations include receiving the image of the cleaning location from the camera and determining a distance from the ultraviolet light source to the cleaning location using the image. Further, the operations include determining a cleaning time period of the cleaning location using the distance from the ultraviolet light source to the cleaning location and controlling the ultraviolet light source to transmit the disinfecting light output for a duration of the cleaning time period.

### CROSS-REFERENCE TO RELATED APPLICATION

This claims the benefit to U.S. Provisional Application No. 63/044,875 filed on Jun. 26, 2020, titled “AMR (Automated Mobile Robot) Using non-mercury Full Spectrum (UVA, UVB, UVC) UV with automated tracking and reporting of cleaning and disinfecting cycles with automated programming for cleaning and disinfecting,” the disclosure of which is hereby incorporated by reference in its entirety for all purposes.

### TECHNICAL FIELD

Examples of the presently disclosed subject matter relate to techniques for cleaning and disinfecting. In particular, the presently disclosed subject matter relates to automated mobile devices used to clean and disinfect areas using ultraviolet light.

### BACKGROUND

The spread of germs, microbes, bacteria, and viruses on surfaces is ubiquitous in numerous industries. Cleaning and disinfecting these surfaces may include costs and inefficiencies associated with human cleaning. Further, documenting and verifying the human cleaning process may introduce additional costs and inefficiencies associated with the cleaning process. Additionally, non-automated ultraviolet (UV) light disinfecting solutions lack automated mobility to provide effective and efficient cleaning of a space while also providing documentation and verification of a cleaning process.

### SUMMARY

Systems and methods for providing automated mobile cleaning and disinfecting are provided.

According to various aspects of the present disclosure, a cleaning system includes a mobile base that enables movement of the cleaning system. The cleaning system also includes a camera that captures an image of a cleaning location and an ultraviolet light source that transmits a disinfecting light output toward the cleaning location. Additionally, the cleaning system includes a processor and a memory. The memory includes instructions stored thereon that are executable by the processor to perform operations. The operations include receiving the image of the cleaning location from the camera and determining a distance from the ultraviolet light source to the cleaning location using the image. Further, the operations include determining a cleaning time period of the cleaning location using the distance from the ultraviolet light source to the cleaning location and controlling the ultraviolet light source to transmit the disinfecting light output for a duration of the cleaning time period.

In an additional example, a method includes capturing an image of a cleaning location using a camera of an automated cleaning system. The method also includes determining a distance from an ultraviolet light source of the automated cleaning system to the cleaning location using the image. Further, the method includes determining an ultraviolet light intensity of the ultraviolet light using the distance from the ultraviolet light source to the cleaning location and determining a cleaning time period of the cleaning location using the ultraviolet light intensity and the distance from the ultraviolet light source to the cleaning location. Furthermore, the method includes firing the ultraviolet light source for a duration of the cleaning time period.

In an additional example, an automated cleaning system includes a mobile base that enables movement of the automated cleaning system and a structural support that extends vertically from the mobile base. The structural support includes a first camera on a first side of the structural support. The first camera is able to capture a first image of a first cleaning location to determine a first distance from the structural support to the first cleaning location. The structural support also includes a second camera on a second side of the structural support opposite the first side. The second camera is able to capture a second image of a second cleaning location to determine a second distance from the structural support to the second cleaning location. Further, the structural support includes a first ultraviolet light on the first side of the structural support. The first ultraviolet light is able to transmit a first disinfecting light output toward the first cleaning location for a first cleaning time period that is determined based on the first distance. Furthermore, the structural support includes a second ultraviolet light on the second side of the structural support. The second ultraviolet light is able to transmit a second disinfecting light output toward the second cleaning location for a second cleaning time period that is determined based on the second distance.

These illustrative aspects and features are mentioned not to limit or define the presently described subject matter, but to provide examples to aid understanding of the concepts described in this application. Other aspects, advantages, and features of the presently described subject matter will become apparent after review of the entire application.

### BRIEF DESCRIPTION OF THE DRAWINGS

Aspects and features of the various examples will be more apparent by describing examples with reference to the accompanying drawings, in which:

FIG. 1 is a perspective view of a cleaning system, in accordance with one or more examples.

FIG. 2 is a partially exploded view of the cleaning system of FIG. 1, in accordance with one or more examples.

FIG. 3 is an interior view of a support structure of the cleaning system of FIG. 1, in accordance with one or more examples.

FIG. 4 is a flowchart of a process for disinfecting a cleaning location using the cleaning system of FIG. 1, in accordance with one or more examples.

FIG. 5 is a block diagram of an example of a computing device, in accordance with one or more examples.

### DETAILED DESCRIPTION

While certain examples are described herein, these examples are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

Certain aspects and examples of the disclosure relate to systems and methods used to disinfect surfaces. In particular, the surface disinfecting systems and methods can include a device implementing non-mercury, full-spectrum ultraviolet (UV) technology, such as a pulsed xenon light source. Such a light source may last longer, consume less energy, and be more environmentally friendly than other disinfecting light sources. The UV technology disinfects and eliminates germs, pathogens, and other microbes in a space. Further, a mobile base of the cleaning system enables the disinfection process without having to manually move and relocate the cleaning system.

In an example, the UV technology is implemented in a compact light engine to decrease the size and weight of the device to enable enhanced mobility and access for cleaning. The cleaning system may rely on smart algorithms and programming for automated calculation of optimum cleaning areas and cleaning time periods. In some examples, the cleaning system provides detailed and verifiable reporting of a disinfecting process to validate a cleaning cycle.

Further, the cleaning system may perform inter-device communication to enhance cleaning operations. For example, multiple cleaning systems may operate in a single space by communicating with one another for increased efficiency in completing the cleaning cycle. The multiple cleaning systems can communicate with one another, or with a base control system, to determine an optimal cleaning strategy for an area. The cleaning systems may also communicate with one another a log of locations that have been cleaned to avoid cleaning or disinfecting an area multiple times by multiple cleaning systems.

The cleaning system may move autonomously once the cleaning system has been commissioned for a cleaning area. In an example, a map of the cleaning area may be downloaded to the cleaning system during a commissioning process. In another example, the cleaning system may learn and map a floor plan of the area prior to performing a cleaning process as part of the commissioning process.

The described examples provide systems and methods to disinfect surfaces. While the described techniques are discussed generally for use with an automated mobile system, they are by no means so limited. Rather, examples of the techniques may be used with systems and devices of any type or otherwise as desired.

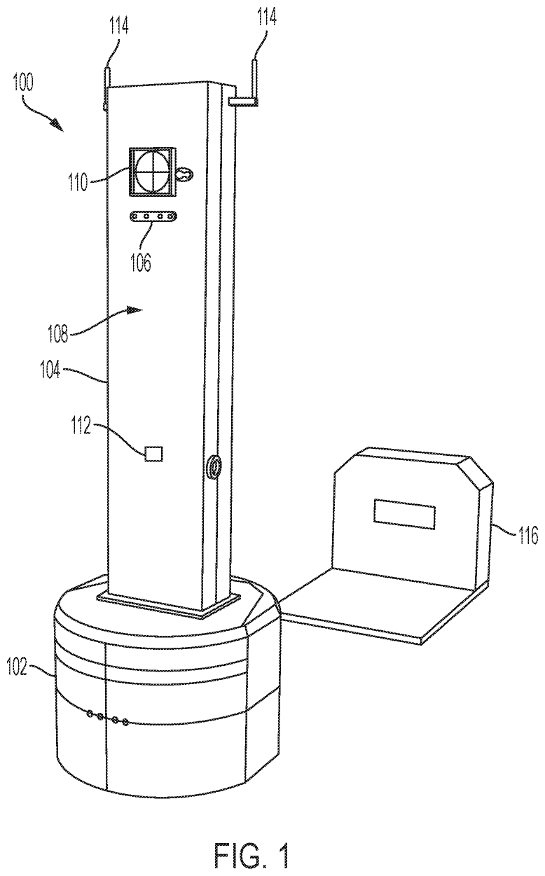

FIG. 1 is a perspective view of a cleaning system 100, in accordance with one or more examples. The cleaning system 100 may include a mobile base 102 that enables movement of the cleaning system 100 between cleaning locations. The mobile base 102 may include a set of wheels powered by a motor. In an example, the cleaning system 100 also includes a structural support 104 extending vertically from the mobile base 102.

In one or more examples, the structural support 104 includes a camera 106 positioned on a first side 108 of the structural support 104. The camera 106 may capture an image of a cleaning location. In some examples, the camera 106 is a three-dimensional camera, and the captured image is a three-dimensional image. The captured image may be used by the cleaning system 100 to determine a distance from the structural support 104 to the cleaning location. In particular, the distance may be determined from an ultraviolet light 110 located on the structural support 104. The ultraviolet light 110 may be positioned one inch above the camera 106 in the same horizontal plane. Other spacing between the ultraviolet light 110 and the camera 106 is also contemplated.

In an example, the ultraviolet light 110 is positioned on the first side 108 of the structural support 104. In operation, the ultraviolet light 110 transmits a disinfecting light output toward the cleaning location to disinfect the cleaning location. The ultraviolet light 110 may include a non-mercury based, full-spectrum ultraviolet light, such as a pulsed xenon light source. In another example, the ultraviolet light 110 may include a far-ultraviolet c (UVC) light. Such an example may operate at a spectrum of 222 nm. Additionally, other ultraviolet light sources capable of disinfecting the cleaning location may also be used as the ultraviolet light 110.

In an example, the ultraviolet light 110 transmits the disinfecting light output for a duration of a cleaning time period. The cleaning time period may be determined using the distance calculated from the ultraviolet light 110 to the cleaning location. In an example, the cleaning time period is determined using an inverse-square of the distance from the ultraviolet light source to the cleaning location and disinfecting qualities of a particular type of the ultraviolet light 110. The cleaning time period may be any time period that is sufficient for disinfecting the cleaning location using the ultraviolet light 110. The transmission of the disinfecting light output by the ultraviolet light 110 toward the cleaning location for the determined cleaning time period may be referred to as a disinfecting process at the cleaning location.

In some examples, the cleaning system 100 may be commissioned prior to performing a cleaning operation in a cleaning area by downloading a map of the cleaning area to the cleaning system 100. In other examples, commissioning of the cleaning system 100 may involve the cleaning system 100 performing a mapping operation of the cleaning area. That is, the cleaning system 100 may autonomously generate its own map of the cleaning area by autonomously traversing the cleaning area to identify areas, rooms, or stations for disinfecting. The mapping process may also identify locations of charging stations 116 for the cleaning system 100.

In another example, the cleaning system 100 may be commissioned by an operator using a remote control device to control movement of the cleaning system 100 through the cleaning area. As the cleaning system 100 traverses the cleaning area, the cleaning system 100 may map the cleaning area. Additionally, disinfection points of the cleaning area may be assigned by the operator in the map of the cleaning area through a graphical user interface.

Further, an optimization of space algorithm may be implemented by the cleaning system 100 or by a centralized computing system (not shown) to limit time and a number of cleaning systems 100 used to disinfect all areas of the cleaning area. This may greatly improve cleaning efficiency when compared to other existing disinfecting technologies. The optimization of space algorithm may also be implemented to conserve battery power of the cleaning system 100. For example, the optimization of space algorithm may provide a cleaning course for the cleaning system 100 that is defined to both optimize cleaning time and minimize battery power consumption. In some examples, an operator of the cleaning systems 100 may provide dates and times for completing a disinfecting process when the cleaning locations are expected to be vacant. Upon completion of the disinfecting process at the cleaning location, the mobile base 102 may autonomously move from the disinfected cleaning location to a new cleaning location based on this optimization of space algorithm.

The cleaning system 100 may also include a motion detector 112. The motion detector 112 may detect motion of an object, such as a human, within a sensitivity range of the motion detector 112. If motion is detected during a disinfecting process, the cleaning system 100 may control the ultraviolet light 100 to stop transmitting the disinfecting light output. In some examples, a speaker (not shown) of the cleaning system 100 may transmit an announcement to an unexpected occupant that they should exit the area due to the ongoing disinfecting processes. When no further motion is detected for a set period of time, such as 15 seconds, the cleaning system 100 may control the ultraviolet light 100 to continue or restart the disinfecting process.

In an example, the cleaning system 100 includes antennas 114 that provide a mechanism to wirelessly communicate with a central computing system or with other cleaning systems 100. For example, the central computing system may provide the cleaning system 100 with instructions for carrying out the disinfecting process or any other communications wirelessly using the antennas 114. In another example, the cleaning system 100 may wirelessly communicate with other cleaning systems 100 using the antenna 114 to coordinate disinfecting workflows. For example, the cleaning systems 100 may communicate with another cleaning system 100 to provide indications of areas that have already been disinfected. Using this information, the cleaning systems 100 may ensure that overlap of disinfecting processes does not occur.

In some examples, a cleaning system 100 may experience a delay in the disinfecting processes due to the presence of occupants in a particular area, as identified with the motion detector 112. In such an example, the cleaning system 100 may communicate with other cleaning systems 100 using the antennas 114. Based on this communication between the cleaning systems 100, a particular cleaning system 100 that is ahead of schedule may compensate for the cleaning system 100 that is behind schedule due to the delay.

In other examples, the cleaning system 100 may communicate with central building control systems associated with the cleaning areas using the antennas 114. In such an example, the communications from the cleaning system 100 may provide a building control system with information used to ignore, for example, movement of the cleaning system 100 through the cleaning area. This may prevent motion sensitive lights within the cleaning area from responding to movement of the cleaning system 100.

In an additional example, the cleaning system 100 may receive a request from a user to clean a particular area. The request may be an “on demand” request when a particular circumstance merits the request. For example, a nurse or doctor in a hospital may request the cleaning system 100 to disinfect a hospital room upon discharge of a patient. In some examples, a computing system or scheduling system may automatically send a request to the cleaning system 100 to perform an additional cleaning of a particular area. That request may be prompted automatically by a triggering event, such as a patient being discharged by the hospital.

In another example, the building control system may coordinate disinfecting processes of elevators with the cleaning system 100. For example, the building control system may disable a particular elevator car when the cleaning system 100 is performing a disinfecting process within the elevator car. Further, the cleaning system 100 may send a request for the building control system to send an elevator car to a particular floor so that the cleaning system 100 can enter the elevator car to conduct a disinfecting process. Other building control coordination between the cleaning system 100 and the building control system are also contemplated, such as door control and lock control to provide the cleaning system 100 access to certain areas. Further, the cleaning system 100 may coordinate operations with any other relevant internet of things (IoT) devices.

Upon completion of disinfecting an assigned cleaning area, or upon a reduction in battery storage to the point where a recharge is necessary, the cleaning system 100 may return to a charging station 116. The charging station 116 may electrically couple with the cleaning system 100 to charge batteries within the cleaning system 100 for use in subsequent disinfecting processes. In an example, the cleaning system 100 may be deployed overnight to disinfect the cleaning area. In such an example, the cleaning system 100 may remain docked at the charging station 116 during standard business hours of the cleaning area until a subsequent overnight cleaning period begins.

In one or more examples, the cleaning system 100, as part of the disinfecting process, can generate a report or a cleaning log that can be used for disinfection verification. For example, the report can verify areas that are cleaned by the cleaning system 100 and effectiveness of a disinfecting process based on a distance between a cleaning location and the ultraviolet light 110 and a transmission time of the disinfecting light output. The report can also include photographs of the cleaning areas, such as work stations, accessed and disinfected by the cleaning system 100. The report may also include information related to occupancy of cleaning areas. Additionally, the report may include information relating to the cleaning system 100 itself, such as remaining life of batteries, light engines (e.g., the ultraviolet lights 110), or other components of the cleaning system 100. Other information collected by the cleaning system 100 may also be included in the report.

In some examples, the cleaning system 100 may also include a radio-frequency identification (RFID) scanner for inventory and price check operations within a retail location. The RFID scanner may also be used for any other RFID applications. In an example, the RFID scanner may be able to log RFID tagged cleaning areas as having been disinfected by the cleaning system 100. The cleaning system 100 may also include an ambient air quality monitoring (AQM) sensor capable of detecting elevated levels of carbon monoxide and carbon dioxide in a cleaning location. Further, the cleaning system 100 may include a security camera that is capable of storing security footage locally within the cleaning system 100 or remotely in cloud storage.

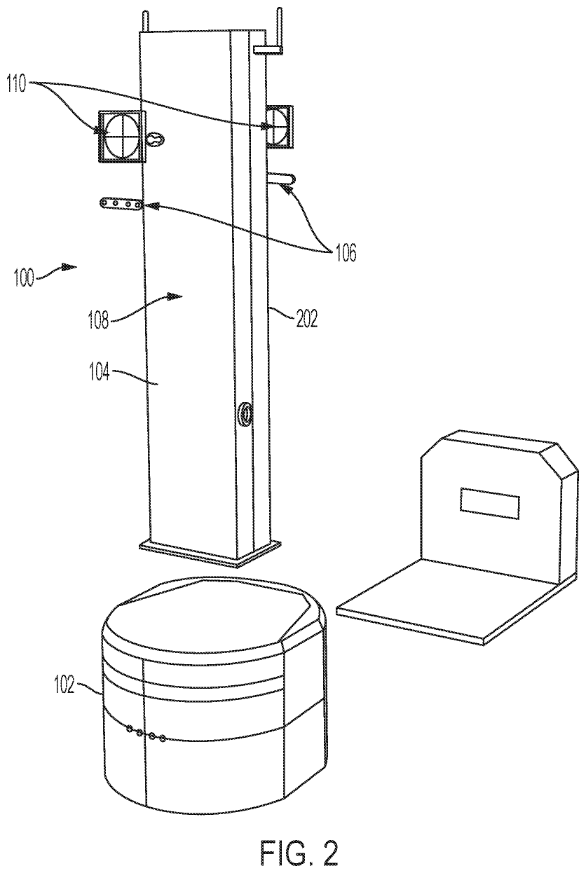

FIG. 2 is a partially exploded view of the cleaning system 100, in accordance with one or more examples. In some examples, the cleaning system 100 can include multiple ultraviolet lights 110 and multiple cameras 106. For example, the support structure 104 may include a second ultraviolet light 110 and a second camera 106 located on a side 202 of the support structure 104 that is opposite the side 108. In some examples, the support structure 104 may include additional sides that support additional ultraviolet lights 110 and cameras 106. For example, the support structure 104 may include four sides where each of the four sides include an ultraviolet light 110 and a camera 106.

When the cleaning system 100 transitions from one cleaning location to a subsequent cleaning location, the cleaning system 100 may rotate such that the cleaning system 100 alternates use of the ultraviolet lights 110. For example, the ultraviolet light 110 on the side 108 of the support structure 104 may be used to disinfect a first cleaning location, while the ultraviolet light 110 on the side 202 may be used to disinfect a second cleaning location. By rotating the ultraviolet lights 110 used in the disinfecting processes, the cleaning system 100 provides the ultraviolet lights 110 with downtime to cool down. This may increase efficiency and longevity of the lights as compared to cleaning systems with individual UV lights by enabling an appropriate amount of cool down time while still performing disinfecting processes. In some addition examples, such as in an elevator car, the cleaning system 100 may transmit the disinfecting light output from both of the ultraviolet lights 110 to speed up a disinfecting process.

In some examples, the mobile base 102 may be removed from the cleaning system 100. In such an example, the support structure 104 may be moved between cleaning locations using other means, such as being carried by an operator. In such an example, the operator may place the support structure 104 toward a center of the cleaning location, and the cleaning system 100 may calculate relevant cleaning time periods based on the location of the support structure 104 in relation to cleaning surfaces.

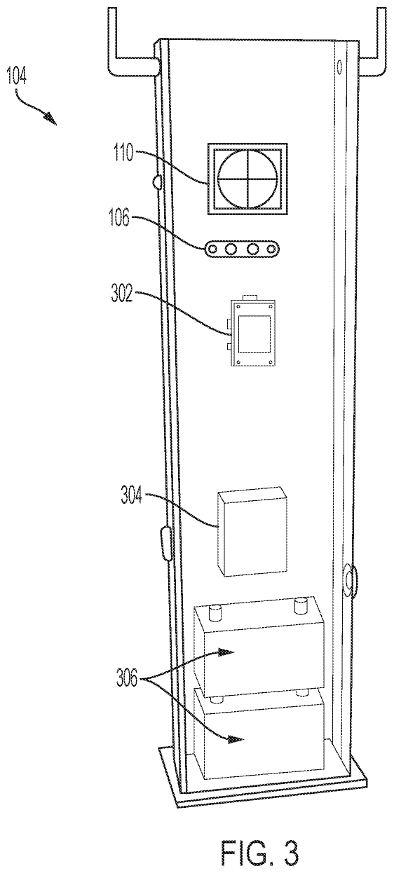

FIG. 3 is an interior view of the support structure 104 of the cleaning system 100, in accordance with one or more examples. In one or more examples, the ultraviolet light 110 may be an LED ultraviolet light. The LED ultraviolet light may provide the cleaning system 100 with the ability to vary the strength of the disinfecting light output. For example, changing a duty cycle of the LED ultraviolet light may provide the cleaning system 100 with the ability to significantly vary the disinfecting light output based on situational details detected by the camera 106, such as a distance from the ultraviolet light 110 to the cleaning area. In other examples, the ultraviolet light 110 may include a fixed intensity. In still other examples, the ultraviolet light 110 may include two or more light engines that enable the ultraviolet light 110 to be fired at two or more different light intensities (e.g., 50% and 100%).

Also included within the support structure 104 is an Ethernet switch 302. The Ethernet switch 302 may route communication traffic from an onboard computer 304 to the components of the cleaning system 100. For example, the Ethernet switch 302 may route lighting control traffic from the onboard computer 304 to the ultraviolet light 110 and camera control traffic from the onboard computer 304 to the camera 106.

Also included within the support structure 104 is a set of batteries 306. The batteries 306 may provide power to all of the devices on the cleaning system 100, and the batteries 306 may provide power to the mobile base 102 described above with respect to FIG. 1. In some examples, the optimization of space algorithm, which is used to control the positioning of the cleaning system 100 with respect to cleaning locations, may also conserve battery power of the batteries 306. In other words, the optimization of space algorithm may also optimize the battery power from the batteries 306 used by the cleaning system 100. While the batteries 306, the onboard computer 304, and the Ethernet switch 302 are all shown within the support structure 104, these components of the cleaning system 100 may be positioned in any other location of the cleaning system 100, such as within the mobile base 102.

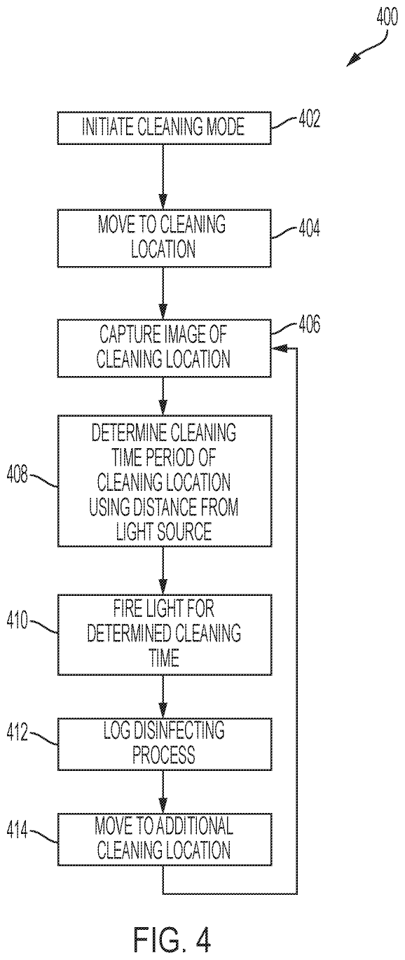

FIG. 4 is a flowchart of a process 400 for disinfecting a cleaning location using the cleaning system 100, in accordance with one or more examples. At block 402, the process 400 involves initiating a cleaning mode of the cleaning system 100. Initiating the cleaning mode may involve commissioning the cleaning system 100 if the cleaning system 100 has not cleaned a particular area before. For example, a map of the area may be downloaded to the cleaning system 100, or the cleaning system 100 may perform an automated mapping process. Additionally, the cleaning system 100 may confirm or receive cleaning instructions. For example, the cleaning system 100 may confirm or receive an indication of a particular portion of a cleaning area to which the cleaning system 100 has been assigned.

At block 404, the process 400 involves moving the cleaning system 100 to a cleaning location. In an example, the cleaning locations can be selected based on a likelihood that those cleaning locations will be in contact with germs, pathogens, and other microbes. For example, a desk, computing station, door handle, etc. may have a high likelihood of being in contact with germs, pathogens, and other microbes. Accordingly, the cleaning system 100 will be programmed to perform a disinfecting process at the desk, computing station, or door handle by identifying that area as a cleaning location. Conversely, a portion of an empty wall within a room with limited foot traffic may not generally be exposed to germs, pathogens, and other microbes. Accordingly, the cleaning system 100 will be programmed to not perform a disinfecting process at the that location. In some examples, the cleaning system 100 is moved as close as possible to the cleaning location due to obstacles that may be positioned between the cleaning system 100 and the cleaning location, such as a chair or table.

In an example where the path to the cleaning location is blocked or where the motion detector 112 is triggered for a predetermined period of time, the cleaning system 100 may proceed to a subsequent cleaning location. Prior to moving to the subsequent cleaning location, the cleaning system 100 may take a picture with the camera 106 to log the reason for not completing the disinfecting process at the particular cleaning location. In some examples, the cleaning system 100 may return to the particular cleaning location to perform the previously bypassed disinfecting process upon completion of the disinfecting process at the subsequent cleaning location or upon completion of the remainder of the disinfecting processes in the cleaning area.

At block 406, the process 400 involves capturing an image of the cleaning location. In an example, the image is captured by the camera 106. In an example, the camera 106 may be a three-dimensional camera that is able to capture a three-dimensional image of the cleaning location. The image may be used to determine a distance between the ultraviolet light 110 and the cleaning location.

At block 408, the process 400 involves determining a cleaning time period of the cleaning location using the distance between the ultraviolet light 110 and the cleaning location. The cleaning time period may also be based on the light intensity output by the ultraviolet light 110. For example, a greater light intensity may result in a shorter cleaning time period than a lesser light intensity. Further, the cleaning time period may be calculated using an inverse-square of the distance from the ultraviolet light 110 to the cleaning location. The cleaning time period may be recalculated every time the cleaning system 100 cleans a particular cleaning location. For example, if an obstacle is between the cleaning system 100 and the cleaning location, the cleaning time period may have to account for the increased distance between the cleaning system 100 and the cleaning location due to the obstacle.

Further, the cleaning time period may also be determined based on an ultraviolet light intensity of the ultraviolet light 110. For example, the distance between the ultraviolet light 110 and the cleaning location may indicate that a particular ultraviolet light intensity may be best suited for a particular disinfecting process. The variable ultraviolet light intensity may have a direct impact, along with the distance between the ultraviolet light 110 and the cleaning location, on determining the cleaning time period.

At block 410, the process 400 involves firing the ultraviolet light 110 for the determined cleaning time period. By firing the ultraviolet light 110, a disinfecting light output is transmitted toward the cleaning location. The disinfecting light output may be maintained by the ultraviolet light 110 for 1.5 to 3 minutes, or however long the cleaning time period indicates the disinfecting light output should be maintained.

At block 412, the process 400 involves logging the disinfecting process. Logging the disinfecting process may include logging a presence of the cleaning system 100 at the cleaning location, logging a cleaning duration performed at the cleaning location, and logging the image captured by the camera 106 of the cleaning location. The log may also include information relating to the cleaning system 100 itself, such as remaining life of the batteries 306, remaining life of the light engines (e.g., the ultraviolet lights 110), or any status information of any other components of the cleaning system 100.

Other information collected by the cleaning system 100 may also be included in the log generated at block 412. For example, the cleaning system 100 may be used in a disinfecting process of produce, meats, dairy, or other food for prolonged shelf life within a food warehouse or storage facility. In such an example, the log may record photos or videos that track the disinfecting process within the food warehouse or storage facility. Further, the log may use the photos or videos for inventory tracking or other IoT related functions.

At block 414, the process 400 involves moving the cleaning system 100 to an additional cleaning location. The cleaning system 100 may be moved upon completion of the disinfecting process (e.g., block 410) and the logging process (e.g., block 412). Once the cleaning system 100 is moved to the additional cleaning location, the process 400 involves capturing an image of the additional cleaning location at block 406, and the remainder of the process 400 may be repeated at the additional cleaning location. Further, in some examples, the cleaning system 100 may be rotated between cleaning locations such that the ultraviolet lights 110 are alternated.

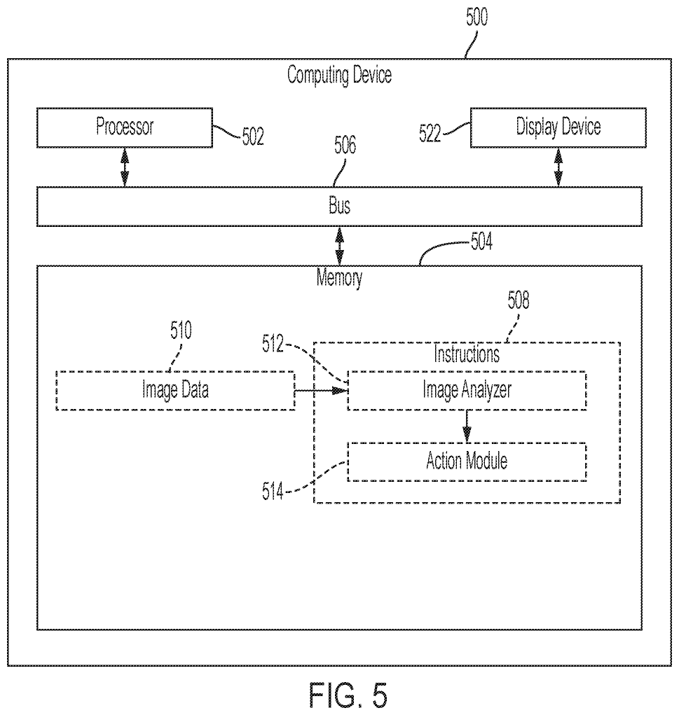

FIG. 5 is a block diagram of an example of a computing device 500, in accordance with one or more examples. While FIG. 5 depicts the computing device 500 as including certain components, other examples may involve more, fewer, or different components than are shown in FIG. 5.

As shown, the computing device 500, which may represent the onboard computer 304, includes a processor 502 communicatively coupled to a memory 504 by a bus 506. The processor 502 can include one processor or multiple processors. Non-limiting examples of the processor 502 include a Field-Programmable Gate Array (FPGA), an application-specific integrated circuit (ASIC), a microprocessor, or any combination of these. The processor 502 can execute instructions 508 stored in the memory 504 to perform operations. In some examples, the instructions 508 can include processor-specific instructions generated by a compiler or an interpreter from code written in any suitable computer-programming language, such as C, C++, C#, or Java.

The memory 504 can include one memory device or multiple memory devices. The memory 504 can be non-volatile and may include any type of memory device that retains stored information when powered off. Non-limiting examples of the memory 504 include electrically erasable and programmable read-only memory (EEPROM), flash memory, or any other type of non-volatile memory. At least some of the memory device includes a non-transitory computer-readable medium from which the processor 502 can read the instructions 508. A non-transitory computer-readable medium can include electronic, optical, magnetic, or other storage devices capable of providing the processor 502 with the instructions 508 or other program code. Non-limiting examples of a non-transitory computer-readable medium include magnetic disk(s), memory chip(s), ROM, random-access memory (RAM), an ASIC, a configured processor, optical storage, or any other medium from which a computer processor can read the instructions 508.

The memory 504 may include image data 510 that is received from the camera 106 positioned on the cleaning system 100. In an example, the image data 510 may be a visual representation of the cleaning location that was captured by the camera 106 prior to a disinfecting process. The instructions 508 may include an image analyzer 512 that can receive the image data 510 as input and provide an output indicating a distance between the ultraviolet light 110 and the cleaning location.

The instructions 508 can also include an action module 514. The action module 514 can include executable program code for taking one or more actions based on the output of the image analyzer 512. For example, the computing device 500 can execute the action module 514 to calculate a cleaning time period based on the distance between the ultraviolet light 110 and the cleaning location. Additionally, the computing device 500 can execute the action module 514 to control the disinfecting light output from the ultraviolet light 110. In additional examples, the action module 514 may be executed by the computing device 500 to communicate with other cleaning systems 100 to coordinate cleaning and disinfecting efforts in a cleaning area. Other actions described herein may also be performed as a result of the computing device 500 executing the action module 514. In some examples, a display device 522 of the computing device 500 may output a graphical user interface (GUI) that outputs status updates relating to the cleaning system 100.

The subject matter of the presently disclosed examples is described herein with specificity to meet statutory requirements, but this description is not necessarily intended to limit the scope of the presently disclosed subject matter. The disclosed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

The foregoing is provided for purposes of illustrating, explaining, and describing various examples. Having described these examples, it will be recognized by those of skill in the art that various modifications, alternative constructions, and equivalents may be used without departing from the spirit of what is disclosed. Different arrangements of the components depicted in the drawings or described above, as well as additional components and steps not shown or described, are possible. Certain features and subcombinations of features disclosed herein are useful and may be employed without reference to other features and subcombinations. Additionally, a number of well-known processes and elements have not been described in order to avoid unnecessarily obscuring the examples. Examples have been described for illustrative and not restrictive purposes, and alternative examples will become apparent to readers of this patent. Accordingly, examples are not limited to those described above or depicted in the drawings, and various modifications can be made without departing from the scope of the presently disclosed subject matter.

That which is claimed is:

1. A cleaning system comprising:
a mobile base configured to enable movement of the cleaning system;
a camera configured to capture an image of a cleaning location;
an ultraviolet light source configured to transmit a disinfecting light output toward the cleaning location;
a processor; and
a memory comprising instructions stored thereon, wherein the instructions are executable by the processor to perform operations comprising:
receiving the image of the cleaning location from the camera;
determining a distance from the ultraviolet light source to the cleaning location using the image;
determining a cleaning time period of the cleaning location using the distance from the ultraviolet light source to the cleaning location; and
controlling the ultraviolet light source to transmit the disinfecting light output for a duration of the cleaning time period.

2. The cleaning system of claim 1, wherein the instructions are further executable by the processor to perform operations comprising:
controlling the mobile base to move to an additional cleaning location;
receiving an additional image of the additional cleaning location using the camera of the cleaning system;
determining an additional distance from the ultraviolet light source to the additional cleaning location using the additional image;
determining an additional cleaning time period of the additional cleaning location using the additional distance from the ultraviolet light source to the additional cleaning location; and
controlling the ultraviolet light source to transmit the disinfecting light output for an additional duration of the additional cleaning time period.

3. The cleaning system of claim 1, wherein the camera comprises at least one three-dimensional camera.

4. The cleaning system of claim 1, wherein the ultraviolet light source comprises a non-mercury based, full-spectrum ultraviolet light.

5. The cleaning system of claim 1, wherein the cleaning time period is determined using an inverse-square of the distance from the ultraviolet light source to the cleaning location.

6. The cleaning system of claim 1, further comprising:
an additional camera;
an additional ultraviolet light source configured to transmit an additional disinfecting light output; and
a structural support extending from the mobile base, wherein (i) the camera and the ultraviolet light source are positionable on a first side of the structural support and (ii) the additional camera and the additional ultraviolet light source are positionable on a second side of the structural support that is opposite the first side, and wherein the instructions stored on the memory are further executable by the processor to perform operations comprising:
controlling the mobile base to move to an additional cleaning location;
receiving an additional image of the additional cleaning location using the additional camera of the cleaning system;
determining an additional distance from the additional ultraviolet light source to the additional cleaning location using the additional image;
determining an additional cleaning time period of the additional cleaning location using the additional distance from the additional ultraviolet light source to the additional cleaning location; and
controlling the additional ultraviolet light source to transmit the disinfecting light output for an additional duration of the additional cleaning time period.

7. The cleaning system of claim 1, wherein the instructions stored on the memory are further executable by the processor to perform operations comprising:
generating a log indicating a presence of the cleaning system at the cleaning location, a cleaning duration performed at the cleaning location, and the image of the cleaning location.

8. The cleaning system of claim 1, further comprising:
an additional ultraviolet light source configured to transmit an additional disinfecting light output in a direction opposite the disinfecting light output from the ultraviolet light source.

9. The cleaning system of claim 8, wherein the mobile base is configured to rotate between the cleaning location and an additional cleaning location such that the additional ultraviolet light source is positionable to transmit the additional disinfecting light output toward the additional cleaning location.

10. A method, comprising:
capturing an image of a cleaning location using a camera of an automated cleaning system;
determining a distance from an ultraviolet light source of the automated cleaning system to the cleaning location using the image;
determining an ultraviolet light intensity of the ultraviolet light using the distance from the ultraviolet light source to the cleaning location;
determining a cleaning time period of the cleaning location using the ultraviolet light intensity and the distance from the ultraviolet light source to the cleaning location; and
firing the ultraviolet light source for a duration of the cleaning time period.

11. The method of claim 10, further comprising:
autonomously moving the automated cleaning system to an additional cleaning location using a mobile base of the automated cleaning system;
capturing an additional image of the additional cleaning location using the camera of the automated cleaning system;
determining an additional distance from the ultraviolet light source to the additional cleaning location using the additional image;
determining an additional cleaning time period of the additional cleaning location using the additional distance from the ultraviolet light source to the additional cleaning location; and
firing the ultraviolet light source for an additional duration of the additional cleaning time period.

12. The method of claim 10, further comprising:
autonomously moving the automated cleaning system to an additional cleaning location using a mobile base of the automated cleaning system;
rotating the mobile base such that an additional camera and an additional ultraviolet light source of the automated cleaning system face the additional cleaning location;
capturing an additional image of the additional cleaning location using the additional camera of the automated cleaning system;
determining an additional distance from the additional ultraviolet light source to the additional cleaning location using the additional image;
determining an additional cleaning time period of the additional cleaning location using the additional distance from the additional ultraviolet light source to the additional cleaning location; and
firing the additional ultraviolet light source for an additional duration of the additional cleaning time period.

13. The method of claim 10, further comprising:
generating a log indicating a presence of the automated cleaning system at the cleaning location, a cleaning duration performed at the cleaning location, and the image of the cleaning location.

14. The method of claim 10, further comprising:
automatically mapping a cleaning area comprising the cleaning location using a mobile base of the automated cleaning system.

15. The method of claim 10, further comprising:
wirelessly communicating with an additional automated cleaning system to coordinate a cleaning workflow of a cleaning area comprising the cleaning location.

16. The method of claim 10, further comprising:
receiving a request to disinfect the cleaning location; and
autonomously moving the cleaning system to the cleaning location.

17. An automated cleaning system, comprising:
a mobile base configured to enable movement of the automated cleaning system;
a structural support positionable to extend vertically from the mobile base, the structural support comprising:
a first camera positionable on a first side of the structural support, wherein the first camera is configured to capture a first image of a first cleaning location to determine a first distance from the structural support to the first cleaning location;
a second camera positionable on a second side of the structural support opposite the first side, wherein the second camera is configured to capture a second image of a second cleaning location to determine a second distance from the structural support to the second cleaning location;
a first ultraviolet light positionable on the first side of the structural support, wherein the first ultraviolet light is configured to transmit a first disinfecting light output toward the first cleaning location for a first cleaning time period that is determined based on the first distance; and
a second ultraviolet light positionable on the second side of the structural support, wherein the second ultraviolet light is configured to transmit a second disinfecting light output toward the second cleaning location for a second cleaning time period that is determined based on the second distance.

18. The automated cleaning system of claim 17, wherein the mobile base is configured to autonomously move from the first cleaning location to the second cleaning location upon completion of the first cleaning time period.

19. The automated cleaning system of claim 17, wherein the first ultraviolet light and the second ultraviolet light are non-mercury based, full-spectrum ultraviolet lights.

20. The automated cleaning system of claim 17, further comprising:
a motion detector configured to detect movement within a vicinity of the motion detector and to disable a disinfecting process of the automated cleaning system upon detecting the movement within the vicinity of the motion detector.
